(19) United States
(12) Patent Application Publication
(10) Pub. No.: US 2008/0299598 A1
(43) Pub. Date: Dec. 4, 2008

(54) Detector Comprising a Membrane Perturbation - Detecting Polymer and Functional Membrane Fragments

(76) Inventor: Raz Jelinek, Reut (IL)

Correspondence Address: NIXON & VANDERHYE, PC, 901 NORTH GLEBE ROAD, 11TH FLOOR, ARLINGTON, VA, 22203, US

(21) Appl. No.: 11/666,134

(22) PCT Filed: Oct. 16, 2005
(86) PCT No.: PCT/IL05/01090

§ 371 Date: Feb. 15, 2008

(30) Foreign Application Priority Data
Oct. 25, 2004 (IL) 168423

(51) Int. Cl. C12Q 1/02; G01N 21/64; G01N 33/566; G01N 21/78

(52) U.S. Cl. 435/29; 436/164; 436/172; 436/501

(57) ABSTRACT

A construct comprising functional membrane fragments and one or more perturbation-detecting polymers associated therewith, wherein said construct responds to perturbations of said membrane fragments by means of a detectable change in one or more physical or chemical properties associated with said construct.

### FIELD OF THE INVENTION

The present invention relates to a calorimetric and or/fluorescent detector comprising a membrane perturbation-detecting polymer and functional membrane fragments, exhibiting a visible color change or a characteristic fluorescence emission in response to the presence of agents and/or conditions capable of causing membrane perturbation.

### BACKGROUND OF THE INVENTION

There currently exist a number of different probes that may be used for investigating physiological and pharmacological events and/or ligands and other factors that cause changes in the properties of biological membranes in vivo and in vitro. Most of these probes, and the techniques associated therewith are limited in their applicability to the measurement of changes in specific ligands or molecular pathways.

There is, however, a need for techniques that can enable real-time reporting of biological or chemical events that may affect the function and/or morphology of either the cell membrane, or alternatively, the function, morphology and viability of the whole cell. A key feature of these desired techniques is that they will not be limited to any specific molecule, molecular pathway or specific cell type or cell line.

A number of prior art publications disclose the use of polydiacetylene-based means for detecting membrane-perturbing events and/or agents:

WO 98/39632 suggests the use of polydiacetylenes for detecting reactions, by means of exposing the reaction means to a biopolymeric material comprising said polydiacetylenes. Preferably, the biopolymeric material is provided in the form of liposomes, films, tubules and other membrane-simulating forms.

WO 99/10743 describes the encapsulation of polydiacetylenes into metal oxide glass, and the use of the transparent composite obtained for the detection of various analytes.

WO 00/55623 discloses a beneficial combination of polydiacetylenes, lipids and suitable means linked thereto for detecting the presence of analytes in a liquid sample, wherein said analytes cannot react chemically with said polydiacetylenes and lipids. Specifically mentioned analytes include metal ions, biological ligands and peptides.

Co-owned, co-pending Israeli patent application no. 158173 discloses a chemical construct comprising polydiacetylenes and lipids which may be assembled within the cell membrane of living cells, wherein said construct retains its ability to undergo its characteristic chromatic transition and fluorescent emission in live cells in response to various perturbing agents and conditions.

It is to be noted, however, that none of the aforementioned documents discloses or describes the construction or use of isolated functional membrane fragments comprising a membrane-perturbation detector such as a polydiacetylene. A need exists for such “ready-made” membrane fragment constructs that may be used in a variety of pharmacological and physiological applications, including drug delivery, drug discovery, and receptor reconstitution.

It is therefore a primary purpose of the present invention to provide a functional membrane fragment based system capable of detecting the presence of agents and conditions causing functional and/or structural membrane perturbation.

It is another purpose of the invention to provide a method for preparing the aforementioned functional membrane fragment based detection system.

Further objects and advantages of the present invention will become apparent as the description proceeds.

### SUMMARY OF THE INVENTION

It has now been found that it is possible to incorporate functional membrane fragments into a membrane perturbation-detecting polymer, such as polydiacetylene, such that the characteristic chromatic and fluorescent changes of said polymer are readily observable when said functional membrane fragments are exposed to agents or conditions that cause alterations in membrane function or structure.

The present invention is primarily directed to constructs comprising functional membrane fragments and one or more perturbation-detecting polymers associated therewith, wherein said constructs respond to perturbations of said membrane fragments by means of a detectable change in one or more physical or chemical properties associated with said construct.

The term “functional membrane fragments” is intended to indicate that said membrane fragments retain, at least partially, the physiological and biochemical properties of the native membrane from which said fragments originated, as can be easily verified using techniques well known in the art. Many different techniques may be used, including, for example, the determination of plasma membrane phosphodiesterase activity (as described in: Gasmi, L. et al., Biochimica et biophysica acta (1998) 1405: 121-7; Chatterjee, S. K. et al. Brit. J. Cancer (1976) 33: 15-26; Braunagel, S. C. et al. Biochimica et biophysics acta (1985) 813: 183-94) and the determination of 5′-nucleotidase activity in plasma membrane (as described in Example 5, hereinbelow and in the following publications: Arkesteijn, C. L. M., J. Clin. Chem. Clin. Biochem. (1976) 14: 155-8; Ekblad L. et al., J. Chromat. B (2000) 743: 397-401; Ming, P. et al. J. Surgical Res. (1997) 69: 418-24.)

The terms “perturbations of the membrane fragments”, “membrane perturbations” and the like are used herein to indicate any changes in the function and/or three-dimensional conformation of said membranes and membrane fragments. The functional changes include, for example, the various biochemical changes that may occur within a membrane following interaction of a molecule with the outer membrane surface. Such changes include the release of second messengers and the subsequent activation of various chemical pathways. Included within this definition are perturbations of the membrane that may lead to the aforementioned changes in function thereof, even when unaccompanied by any detectable or measurable conformational changes.

The aforementioned physical or chemical property associated with the construct may be any property or function that may be readily detected and/or measured. Preferably, such a property will be a form of electromagnetic radiation, such as visible light or ultraviolet or infrared radiation. Most preferably, the detected property will be a chromatic transition (e.g. a change in visible color of the construct comprising functional membrane fragments and one or more perturbation-detecting polymers) or a fluorescent emission. Consequently, in one preferred embodiment of the present invention, the detectable change in the physical or chemical properties associated with the novel constructs is a change in the visible range absorption spectrum thereof. In another such preferred embodiment, the detectable change in the physical or chemical properties is a change in the fluorescent emission spectrum of said construct.

In one preferred embodiment of the present invention, the perturbation-detecting polymer is polydiacetylene (PDA).

In an even more particularly preferred embodiment, the PDA is a polymer of 10,12-tricosadionic acid.

When the polymer incorporated into the aforementioned construct is PDA, the detectable properties associated therewith are a blue-to-red color transition and a fluorescent emission at about 560 or 650 nm emitted from the red phase of the polymer following excitation at ˜490 nm.

Another aspect of the invention relates to a process for preparing constructs comprising functional membrane fragments and one or more perturbation-detecting polymers associated therewith, said process comprising providing functional membrane fragments, mixing said membrane fragments with a solution containing the monomer precursors of one or more perturbation-detecting polymers and exposing the mixture to conditions that permit polymerization of said monomer precursors.

In one preferred embodiment of the process of the invention, the monomer precursors are monomers that may be polymerized to form PDA. In an even more particularly preferred embodiment, the monomer is 10,12-tricosadionic acid. In the case of PDA precursors, polymerization is achieved by means of exposing the mixture of the monomers and membrane fragments to ultraviolet irradiation.

The functional membrane fragments may be obtained from any suitable and convenient source including cultured cells, cells isolated from living tissue and bacteria.

In another aspect, the present invention provides a method for detecting membrane perturbation, comprising contacting a construct comprising functional membrane fragments and one or more perturbation-detecting polymers associated therewith a tested sample, and either observing the color of said construct or obtaining a fluorescent emission thereof, wherein a change in said color or a characteristic fluorescence emission indicate the presence of agents and/or conditions capable of causing membrane perturbation within said tested sample.

All the above and other characteristics and advantages of the present invention will be further understood from the following illustrative and non-limitative examples of preferred embodiments thereof.

### BRIEF DESCRIPTION OF THE DRAWINGS

FIG. 1 illustrates the detection of atropine molecules by the construct of the invention (comprising membrane fragments obtained from PC12 cells over expressing the muscarinic receptor or regular PC12, and polydiacetylene (PDA)) following the interaction of said atropine molecules with said receptor.

FIG. 2 illustrates the detection of a drug molecule capable of permeating cellular membrane using the construct of the invention (comprising plasma membrane fragments obtained from PC12 cells and polydiacetylene).

### DETAILED DESCRIPTION OF PREFERRED EMBODIMENTS

In one particular preferred embodiment of the present invention, the membrane perturbation-detecting polymer is polydiacetylene (PDA). Preferred diacetylene monomers that may be used in the present invention for preparing the chromatic polydiacetylenes are well known in the art and are described, inter alia, in WO 99/10743 and US 2002/0034475, which are all incorporated herein by reference.

Most preferably, the monomers are selected from the group consisting of 10,12-tricosadiynoic acid, 10,12-pentacosadiynoic acid, 10,12-octadecadiynoic acid, 5,7-docosadiynoic acid, 5,7-pentacosadiynoic acid and 5,7-tetracosadiynoic acid. These monomers are all commercially available.

The preparation of the construct of the present invention involves, as a first stage, the formation of an aqueous co-suspension comprising the monomer precursors of the perturbation-detecting polymer together with functional membrane fragments. Typically, polymer precursors, such as the particularly-preferred 10,12-tricosadiynoic acid, are removed from their organic solvent-containing stock solution by means of drying to constant weight and then resuspended in water. The resuspended monomers are then subjected to probe-sonication at an elevated temperature, preferably in the range of 60 to 90° C., most preferably at a temperature of 70° C. The duration of the sonication is dependent on the power generated by the sonicator. For example, for a sonicator of 100 W power, with short intermission between the sonication pulses, the duration of the sonication is between 3 to 5 minutes. Alternatively, the aqueous suspension may be subjected to bath sonication or to extraction through French Press apparatus.

The functional membrane fragments may be obtained from any convenient source, including homogenized tissue samples and cultured primary cells and cell lines. A variety of standard methods may be used to obtain purified plasma membrane fractions; one such method is described in the Example given hereinbelow. Typically, the membrane fragments are transferred to a buffered aqueous suspension at approximately neutral pH prior to their addition to the precursor (monomer) suspension. A particularly preferred buffer is 10 mM Tris-HCl, pH 7. However, for some cell membranes it is not necessary to use a buffer.

Following the formation of the co-suspension at approximately 35-50° C., as described above, said co-suspension is subjected to a short burst of probe sonication, preferably in the range of 5 seconds to 30 seconds, most preferably for 10 seconds, using the conditions described hereinabove.

Preferably, the monomers and membrane fragments are present in the co-suspension at a weight ratio of between 1:1 and 10:1, most preferably at a weight ratio of 5:1.

The volume of aqueous solvent should be such that the concentration of the resulting suspension, calculated according to the total amount of the monomer and membrane fragment, is between 0.5 mM and 5 mM, more preferably between 1 mM and 3 mM.

Following the preparation of the co-suspension of monomers and membrane fragments, said co-suspension is placed in the refrigerator overnight. The co-suspension is then exposed to conditions that cause polymerization of the monomers. In the case of the most preferred embodiment of the present invention, in which the polymer present in the construct is PDA, polymerization is achieved by subjecting the co-suspension to ultraviolet (UV) irradiation, preferably at a wavelength of 254 nm. In this case, prior to irradiation, the co-suspension is allowed to gradually cool down and is maintained at 4° C. for at least 6 hours. The co-suspension is then irradiated at 254 nm for about 10 to 50 seconds, preferably by means of UV oven (cross linker) or UV lamp, to polymerize the diacetylene monomer.

The resulting membrane perturbation-detecting construct comprising the polydiacetylene incorporated into the functional membrane fragments exhibits a blue color and may be easily and conveniently used for rapidly detecting the presence of various analytes that are capable of interacting with cellular membrane, such as microorganisms and toxins produced thereby, metal cations, peptides, proteins, biological ligands and pharmaceutically active compounds. In general, it is sufficient to add the sample to be tested to a suspension of the PDA-membrane construct, and following a suitable incubation period, which depends on the type of the analyte, observing the color of said construct, wherein a change in said color indicates the presence of said analyte in the tested sample. A minimal concentration of the analyte, that is detectable by the method of the invention, is typically in the range of 1 μM to 1 mM. Typical incubation periods may vary between 0.5 to 30 minutes for ligands selected from the group consisting of metal cations, peptides, pharmaceutically active compounds, proteins and other biological ligands.

Metal cations that may be detected according to the present invention include alkali or alkaline-earth metals, as well as transition metals.

Peptides that may be detected by the method of the present invention include antimicrobial peptides, membrane-active peptides and cytolytic peptides. The peptides may contain between 5 to 100 amino acids, and may have hydrophobic and amphipathic domains.

Pharmaceutically active compounds that may be detected by the method of the present invention include, but are not limited to, hydrophobic compounds having molecular weight of below 1000 g/mol, that are capable of binding and permeating cellular membrane or physiological lipid barriers, such as drugs, metabolites and penetration enhancers.

Another important application of the functional membrane fragment-PDA construct of the present invention is the detection and analysis of complex cooperative processes such as signaling events, ligand-receptor interactions, cell-cell communication and interaction between viruses and sites within the cell membrane.

Proteins that may be detected by the method of the present invention include, but are not limited to, membrane proteins, lipophilic enzymes and signaling proteins. Other biological ligands that may be detected by the method of the present invention include hormones and biological compounds that specifically bind or permeate cellular membranes or have specific affinities to membrane receptors.

In addition to the above-described use of the PDA-membrane construct of the present invention in the detection of various analytes that cause functional and/or structural membrane perturbations, said construct is of specific value in the following applications:

### 1. Receptor Reconstitution

The use of functional membrane fragments in the construct of the present invention renders said construct highly suitable for the isolation and reconstitution of cellular receptor proteins. Following receptor reconstitution, the receptor-bearing constructs could then be used as carriers of, for example, hormone molecules.

### 2. Drug Discovery

The presently-claimed constructs may be used for the high throughput screening of drug action on membranes.

### 3. Studying Membrane Interactions

In view of the fact that the presently-claimed construct comprises functional membrane fragments, said construct may be used to study the effect of the surface binding, membrane penetration and/or membrane disruption caused by various molecular species.

### 4. Imaging of Membrane-Related Activities

Since the construct of the present invention includes a colorimetric/fluorometric reporter molecule (i.e. PDA), said construct is ideally suited for real-time visual tracking of agents and events that cause changes in membrane function and/or structure.

### 5. Drug Delivery

An example of the use of this use would be the incorporation of drug molecules within liposomes or other membrane forms comprising the presently-claimed construct.

The blue to red transition exhibited by the PDA-membrane construct can be observed by the naked eye. Alternatively, the color changes may be recorded by means of a UV-vis spectrophotometer or an ELISA plate reader. Typically, the spectrophotometric reading is made at 27° C. using a 1 cm optical path cell with a standard laboratory spectrophotometric device such as the Jasco spectrophotometer. The quantitative measurement of the color transition exhibited by the construct comprising polydiacetylene and functional membrane fragments in the presence of the analyte may be carried out similarly to the description given in WO 00/55623, which is incorporated herein by reference, and as exemplified hereinbelow.

Alternatively, it may be appreciated that in addition to chromatic transition, the constructs according to the present invention may respond to perturbations of the functional membrane fragments comprised therein by means of a characteristic fluorescent emission. Detection of this fluorescent emission may be accomplished by illuminating the novel construct of the invention with a suitable light source emitting light at about 500-505 nm. The appearance of characteristic maxima at about 560 and/or 650 nm in the fluorescence spectrum obtained following said excitation serves as an indication that the functional membrane fragments present in the construct of the invention have been perturbed. The aforementioned procedure may be suitably carried out using an inverted microscope fitted with fluorescent excitation and detection means, or a standard fluorescence spectrophotometer.

The following examples are provided for illustrative purposes and in order to more particularly explain and describe the present invention. The present invention, however, is not limited to the particular embodiments disclosed in the examples.

### EXPERIMENTAL

### Materials and Methods

The diacetylenic monomer 10,12-tricosadiynoic acid was purchased from GFS Chemicals (Powell, Ohio), washed in chloroform, and filtrated through a 0.45 μm filter prior to use.

Tris[hydroxymethyl]aminomethane (Tris-base buffer) (C₄H₁₁NO₃), Tris-acid, phenylmethylsulphonyl fluoride (PMSF) and percoll were purchased from Sigma.

### Cell Culture:

Caco2 cells were obtained from the American Type Culture Collection and cultured in tissue culture flasks containing Dulbecco's minimal essential medium (DMEM) supplemented with 10% fetal bovine serum (FBS), 100 units/ml penicillin and 100 μg/ml streptomycin. The medium was additionally supplemented with 1 mM sodium pyruvate, 10 mM non-essential amino acids and 2 mM L-glutamine. Cells were incubated at 37° C. in a 5% CO₂ atmosphere. The culture medium was changed every alternate day and the cells harvested from their flasks with a trypsin-EDTA solution (0.25% w/v).

PC12 cells overexpressing the muscarinic receptors were prepared using conventional transfection techniques, as described, for example, by Sadot et al. [Israel Journal of neurochemistry (1996 February), 66(2), 877-80.]

### Example 1

### Preparation of Constructs Comprising PDA and Membrane Fragments Obtained from Caco2 Cells

### A. Extraction of Plasma Membrane:

### Preparation of Crude Membrane (CM):

15 ml of cell suspension are centrifuged at 3000 g for 5 min. The harvested cells (1 g wet weight) are resuspended in 2 ml of grinding medium (250 mM sucrose; 10 mM Tris-Cl, pH 7.5; 1 mM Phenylmethylsulphonyl fluoride (PMSF)), to which have been added 3 mm diameter borosilicate glass beads (Aldrich Inc.) and subjected to bath sonication for 5 minutes. Following sonication, the homogenate is centrifuged at 1000 g for 5 minutes at 4° C., to remove unbroken cells and glass beads.

The pellet thus obtained is then washed once with the grinding medium. The supernatant obtained from this washing step is pooled with the supernatant obtained from the previous (1000 g) centrifugation step and re-centrifuged at 15000 g for 40 minutes at 4° C. The supernatant is discarded, the pellet containing the crude membrane is resuspended in suspension buffer (10 mM Tris-Cl, pH 7), and, if not required immediately, stored at −70° C.

### Isolation of Plasma Membrane (PM):

An aliquot of the CM preparation (obtained as described above) containing 25-30 mg protein (as determined by the Lowry method) in 4 ml of suspension buffer is diluted in Percoll (18%, v/v) in 10 mM Tris-Cl, pH 7.5. The suspension thus obtained is then centrifuged at 40000 g for 40 minutes in a SW-41 Ti type Beckman rotor. Following centrifugation, the self-generating Percoll gradient has a translucent lipid layer on the top and a plasma membrane (PM) band just below said lipid layer. The PM layer is then aspirated and washed with 10 mM Tris-Cl, pH 7.5, at 100000 g for 30 minutes in the same rotor. The PM pellet is then resuspended in suspension buffer, and, if not required immediately, stored at −70° C. until further use.

B. Formation of PDA Incorporated within the Membrane Fragments

Preparation of vesicles containing Caco2 plasma membrane fragments and PDA is carried out as follows:

20 μl of 10,12-tricosadionic acid taken from a stock solution in 60 mM chloroform/ethanol (1:1) are dried in vacuo up to constant weight. The dried material is then resuspended in 1.950 ml deionized water and subjected to probe sonication at 70° C. for 3 minutes. The solution thus obtained is then cooled for 15 minutes, and 50 μl of plasma Caco2 cell PM (prepared as described hereinabove and containing 18.5 mg/ml protein) is added thereto. The vesicle suspension obtained thereby is then subject to another round of sonication for 10 seconds, left to cool to room temperature and than stored at 4° C. overnight. The 10,12-tricosadionic acid is then polymerized by means of irradiating the vesicle suspension at 254 nm for 30-40 seconds, at the end of which time, the resulting suspension exhibits a blue appearance.

### Example 2

### Preparation of Constructs Comprising PDA and Membrane Fragments Obtained from PC12 Cells

### A. Extraction of Plasma Membrane:

Preparation of Crude Membrane (CM) from PC12 Cells:

15 ml of cell suspension (containing about 3×10⁶ cells) are centrifuged at 3000 g for 5 min. The harvested cells (1 g wet weight) are resuspended in 2 ml of grinding medium (250 mM sucrose; 10 mM Tris-Cl, pH 7.5; 1 mM Phenylmethylsulphonyl fluoride (PMSF)), and subjected to bath sonication for 5 minutes. Following sonication, the homogenate is centrifuged at 1000 g for 5 minutes at 4° C., to remove unbroken cells.

The pellet thus obtained is then washed once with the grinding medium. The supernatant obtained from this washing step is pooled with the supernatant obtained from the previous (100 g) centrifugation step and re-centrifuged at 15000 g for 40 minutes at 4° C. The supernatant is discarded, the pellet resuspended in suspension buffer (10 mM Tris-Cl, pH 7), and, if not required immediately, stored at −70° C.

Isolation of Plasma Membrane (PM) from PC12 Cells:

An aliquot of the CM preparation (obtained as described above) containing 10-15 mg protein (as determined by the Lowry method) in 2.5-3 ml of suspension buffer is diluted in Percoll (18%, v/v) in 10 mM Tris-Cl, pH 7.5. The suspension thus obtained is then centrifuged at 40000 g for 40 minutes in a SW-55 type Beckman rotor. Following centrifugation, the self-generating Percoll gradient has a translucent lipid layer on the top and a plasma membrane (PM) band just below said lipid layer. The PM layer is then aspirated and washed with 10 mM Tris-Cl, pH 7.5, at 100000 g for 30 minutes in the same rotor.

B. Formation of PDA Incorporated within the Membrane Fragments
(i) Preparation of Vesicles Containing Crude Membrane (CM) from PC12 Cells and PDA:

20 μl of 10,12-tricosadionic acid taken from a stock solution in 60 mM chloroform/ethanol (1:1) are dried in vacuo up to constant weight. The dried material is then resuspended in 1.950 ml deionized water and subjected to probe sonication at 70° C. for 3 minutes. The solution thus obtained is then cooled for 15 minutes, and 50 μl of crude membrane from PC12 cells (prepared as described hereinabove and containing 10 mg/ml protein) is added thereto. The vesicle suspension obtained thereby is then subject to another round of sonication for 10 seconds, left to cool to room temperature and than stored at 4° C. overnight. The 10,12-tricosadionic acid is then polymerized by means of irradiating the vesicle suspension at 254 nm for 30-40 seconds, at the end of which time, the resulting suspension exhibits a blue appearance.

(ii) Preparation of Vesicles Containing Plasma Membrane (PM) from PC12 Cells and PDA:

20 μl of 10,12-tricosadionic acid taken from a stock solution in 60 mM chloroform/ethanol (1:1) are dried in vacuo up to constant weight. The dried material is then resuspended in 1.950 ml deionized water and subjected to probe sonication at 70° C. for 3 minutes. The solution thus obtained is then cooled for 15 minutes, and 50 μl of plasma membrane from PC12 cells (prepared as described hereinabove) is added thereto. The vesicle suspension obtained thereby is then subject to another round of sonication for 10 seconds, left to cool to room temperature and than stored at 4° C. overnight. The 10,12-tricosadionic acid is then polymerized by means of irradiating the vesicle suspension at 254 nm for 30-40 seconds, at the end of which time, the resulting suspension exhibits a blue appearance.

### Example 3

### Use of the Construct Comprising Functional Membrane Fragments and Polydiacetylenes as a Calorimetric Detector

An aqueous stock solution containing Atropine at a concentration of 0.55 mg/ml was prepared. A series of samples containing the blue construct according to the invention (prepared as described hereinabove in Example 2) and atropine at different concentrations (up to 20 μM) were prepared as follows. In each sample, 0.06 mL of the blue construct solution prepared according to Example 2 and 25 mM Tris base (pH 8) solution were mixed with an aliquot taken from the atropine stock solution.

The UV-vis spectroscopic measurements were carried out three times for each of the samples prepared, using a Jena Analytical Elisa-reader using 96 well microplates. The blue-to-red chromatic transitions were quantified using the chromatic response factor, as defined below:

$$\% CR = \frac{{PB}_{0} - {PB}_{1}}{{PB}_{0}} \times 100 , where PB = \frac{{A}_{blue}}{{A}_{blue} + {A}_{red}} ,$$

where A_{blue} and A_{red} are the absorbance measured at about 640 nm and about 500 nm, respectively. PB₀ is the blue/red ratio of the construct solution before induction of color change, and PB_{I} is the value obtained after adding the tested sample thereto.

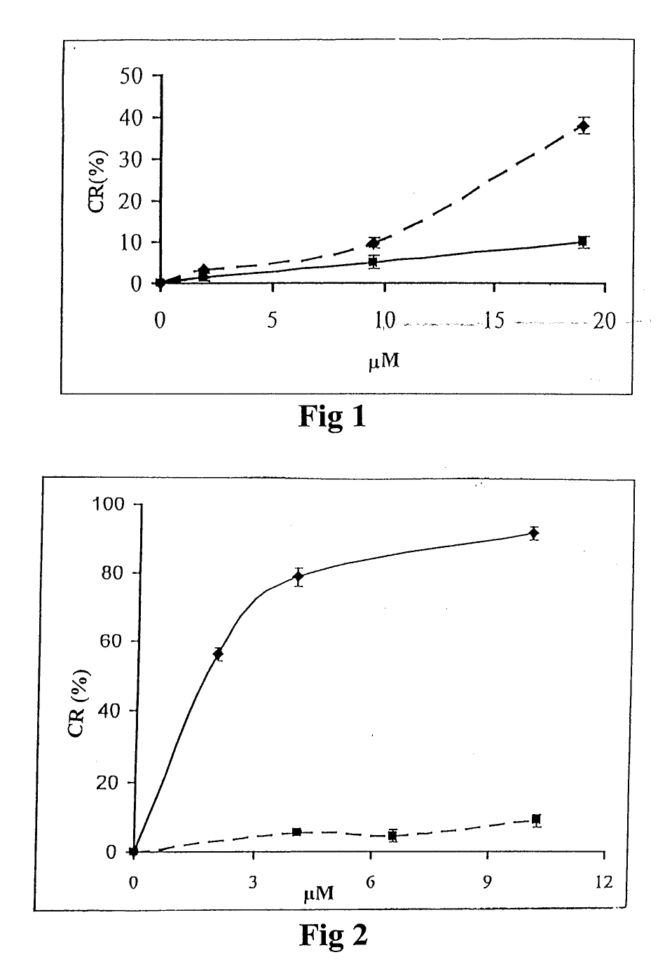

FIG. 1 shows the chromatic response determined for various atropine containing samples on the basis of the UV-vis spectra measured, versus the concentration of atropine in said samples. The solid line corresponds to the results obtained for atropine samples containing a construct made of crude membrane fragments extracted from PC12 cells and polydiacetylene, whereas the broken line indicates the results obtained for atropine samples that contain a construct made of crude membrane fragments extracted from PC₁₂ cells overexpressing muscarinic receptor in their membrane, and polydiacetylene. It is apparent from the figure that the construct according to the invention undergoes a much more significant chromatic transition (high % CR) when said construct comprises membrane fragments obtained from cells overexpressing the muscarinic receptor, which specifically recognize said atropine molecules.

### Example 4

### Use of the Construct Comprising Functional Membrane Fragments and Polydiacetylenes as a Calorimetric Detector

Aqueous stock solutions containing 5.2 mg/ml propranolol and 1.5 mg/ml amoxicillin were prepared. A series of samples containing the blue construct according to the invention (prepared as described hereinabove in Example 2) and either propranolol or amoxicillin at different concentrations (up to 11 μM in both cases) were prepared as follows. In each sample, 0.06 mL of the blue construct solution prepared according to Example 2 and 25 mM Tris base (pH 8) solution were mixed with an aliquot taken from either the propranolol or amoxicillin stock solutions.

The UV-vis spectroscopic measurements and the calculation of the chromatic response factor were carried out as described hereinabove in Example 3.

FIG. 2 depicts the chromatic response factor calculated for various Propranol- or Amoxicillin-containing samples on the basis of the UV-vis spectra measured, versus the concentration of the analyte (that is, either Propranol or Amoxicillin) in said samples. The solid line corresponds to the results obtained for samples containing the construct according to the present invention and Propranol, whereas the broken line indicates the results obtained for samples that contain the construct according to the invention and Amoxicillin. The figure shows that a molecule such as propranolol, which is known to have the capability to strongly interact with the plasma membrane, indeed induces a strong detectable chromatic change (high % CR) in the construct. In contrast, a molecule such as Amoxicillin, which is known to lack the capability to pass through cellular membranes in a passive manner, does not change the color of the construct according to the invention (very low % CR).

### Example 5

### Test for Membrane Functionality

### Determination of 5′-Nucleotidase Activity

5′-Nucleotidase (5′-ND; 5-ribonucleotide phosphohydrolase) is a group of enzymes that specifically hydrolyze 5′-nucleotides, such as adenosine 5′-monophosphate to nucleosides and inorganic phosphorus. Procedures for assaying 5′-ND are based on measuring either the nucleoside or the inorganic phosphorus produced by the hydrolytic action of the enzyme on nucleotide substrate.

5′-ND causes the hydrolysis of adenosine monophosphate (AMP) to yield adenosine and inorganic phosphorus (Pi). The auxiliary enzyme, adenosine deaminase (ADA) deaminates adenosine, producing inosine and ammonium ion (NH₄⁺). In a coupled reaction catalyzed by L-glutamate dehydrogenase (GLDH), the NH₄⁺ reacts with 2-Oxoglutarate in the presence of reduced nicotinamide adenine dinucleotide (NADH) to form glutamate and NAD. The rate of NAD formation, which produced a decrease in absorbance at 340 nm, is directly proportional to the rate of adenosine formation and, hence, 5′-ND activity.

The following test is based on the use of the 5′-ND assay kit provided by Sigma Inc., St. Louis, Mo.:

5′-ND Reagent contains:

| | | |
|---|---|---|
| AMP | 3.2 | mmol/L |
| NADH | 0.2 | mmol/L |
| 2-Oxoglutarate | 3.7 | mmol/L |
| GLDH (bovine) | 11,000 | U/L |
| ADA (bovine) | 400 | U/L |
| β-Glycerophosphate, buffer and stabilizers | | |

### Procedure:

The temperature of the reaction mixture should be maintained at 30° C. or some other constant temperature.
1. Pipet 11.0 ml Assay Solution into a cuvet and bring to reaction temperature
2. Add 0.067 ml sample and mix by inversion
3. Place cuvet in constant temperature cuvet compartment and wait approximately 5 minutes
4. Read and record the absorbance (A) of TEST at 340 nm vs. water as reference. This is INITIAL A
5. Exactly 5 minutes later, again read and record the absorbance. This is FINAL A

### Calculations:

ΔA per 5 min=INITIAL A−FINAL A
5′-ND (U/L)=μA per 5 min*512.07*Temperature correction factor (TCF). TCF (30° C.)=1
Wherein:
   Factor 512.07=(1.067*1000)/(6.22*0.067*5)
1.067=total reaction volume (mL)
1000=conversion of activity per ml to activity per L
6.22=mM absorptivity of NADH at 340 nm
0.067=volume of sample (ml)
5=conversion of ΔA per 5 min to A per min

The membrane fragments are considered functional when the above calculated 5′-ND (U/L) value is preferably above 0.5 U/L.

While specific embodiments of the invention have been described for the purpose of illustration, it will be understood that the invention may be carried out in practice by skilled persons with many modifications, variations and adaptations, without departing from its spirit or exceeding the scope of the claims.

1. A construct comprising functional membrane fragments and one or more perturbation-detecting polymers associated therewith, wherein said construct responds to perturbations of said membrane fragments by means of a detectable change in one or more physical or chemical properties associated with said construct.

2. The construct according to claim 1, wherein the perturbation-detecting polymer is polydiacetylene (PDA).

3. The construct according to claim 2, wherein the PDA is a polymer of a monomer selected from the group consisting of 10,12-tricosadiynoic acid, 10,12-pentacosadiynoic acid, 10,12-octadecadiynoic acid, 5,7-docosadiynoic acid, 5,7-pentacosadiynoic acid and 5,7-tetracosadiynoic acid.

4. A process for preparing constructs comprising functional membrane fragments and one or more perturbation-detecting polymers associated therewith, said process comprising providing functional membrane fragments, mixing said membrane fragments with a solution containing the monomer precursors of one or more perturbation-detecting polymers and exposing the mixture to conditions that permit polymerization of said monomer precursors.

5. The process according to claim 4, wherein the precursors are monomers that may be polymerized to form PDA.

6. The process according to claim 5, wherein the monomer is 10,12-tricosadyionic acid.

7. The process according to claim 5, wherein polymerization is achieved by means of exposing the mixture of monomers and membrane fragments to ultraviolet irradiation.

8. A method for detecting membrane perturbation, comprising contacting a tested sample with a construct comprising functional membrane fragments and one or more perturbation-detecting polymers associated therewith, and either observing the color of said construct or obtaining a fluorescent emission thereof, wherein a change in said color or a characteristic fluorescence emission indicate the presence of agents and/or conditions capable of causing membrane perturbation within the tested sample.

9. A method according to claim 8, wherein the agents present in the sample are capable of interacting with cellular membranes and/or permeating cellular membranes.

10. A method according to claim 9, wherein the agents are selected from the group consisting of microorganisms and toxins produced thereby, metal cations, peptides, proteins, biological ligands and pharmaceutically active compounds.

11. A method according to claim 8, wherein the membrane perturbation detected by the construct is an event selected from the group consisting of binding to the membrane surface, membrane penetration and/or membrane disruption caused by various molecular species present in the tested sample.

12. A method for real-time visual tracking of agents and events that cause membrane perturbations, comprising exposing the construct defined in claim 1 to said agents and/or events and recording either a chromatic transition or a characteristic fluorescent emission spectrum thereof.
